(19) United States
(12) Patent Application Publication
(10) Pub. No.: US 2015/0079142 A1
(43) Pub. Date: Mar. 19, 2015

(54) DEVICES AND METHODS FOR PREVENTING PLATELET ACTIVATION

(71) Applicant: The Children's Hospital of Philadelphia, Philadelphia, PA (US)

(72) Inventors: Stanley J. Stachelek, Philadelphia, PA (US); Matthew J. Finley, Abington, PA (US); Robert J. Levy, Merion Station, PA (US)

(21) Appl. No.: 14/385,890

(22) PCT Filed: Mar. 15, 2013
(86) PCT No.: PCT/US13/32177

§ 371 Date: Sep. 17, 2014

Related U.S. Application Data
(60) Provisional application No. 61/612,618, filed on Mar. 19, 2012.

(51) Int. Cl. A61L 27/54; A61L 27/16; A61L 27/18; A61J 1/14

(52) U.S. Cl. CPC A61L 27/54; A61J 1/14; A61L 27/16; A61L 27/18; A61L 2300/256; A61L 2300/252; A61L 2300/42
USPC 424/422; 427/2.3; 427/2.25; 206/232; 424/130.1; 514/13.8

(57) ABSTRACT

Methods for inhibiting a biomaterial-associated thrombotic event comprise reducing the number of platelets that bind to the biomaterial, or inhibiting platelet activation, by attaching CD47 or the Ig domain thereof to the surface of the biomaterial. Methods of the present invention inhibit thrombi formation on or near a biomaterial that is on the surface of an implant, medical device, tube, or therapeutic delivery vehicle. Also provided are kits for practicing these methods and the modified biomaterials.

### CROSS-REFERENCE TO RELATED APPLICATIONS

This application is a U.S. National Phase Application of PCT International Application No. PCT/US2013/032177, filed Mar. 15, 2013, and claims the benefit of U.S. Provisional Application No. 61/612,618, filed Mar. 19, 2012, which applications are incorporated by reference herein, in their entireties and for all purposes.

### FIELD OF THE INVENTION

This invention relates generally to inhibiting platelet activation and subsequent thrombotic events. In particular embodiments, the invention relates to methods for protecting a biomaterial from thrombi formation.

### BACKGROUND OF THE INVENTION

The use of a biomaterial, such as a cardiovascular device, represents the introduction of a foreign surface into a subject's circulation. Thrombosis is a complication frequently associated with medical devices whereby a thrombus or blood clot forms when a device implanted in the body comes into contact with blood. Interactions between the blood cells and biomaterial can trigger a complex series of events, including platelet activation and adhesion.

Thrombotic responses induced by biomedical devices remain a serious concern in the medical field. Cardiovascular devices, in particular, present serious risks of thrombotic complications and have been to known to cause various types of complications that can have potentially fatal outcomes, such as obstruction of intra-arterial stents, catheters, and prosthetic valves, as well as complications during cardiopulmonary bypass and angioplasty. Thrombotic complications with cardiovascular devices can occur despite the use of anti-platelet therapies. There has been limited success in improving the blood compatibility of cardiovascular devices and there is a significant unmet medical need for ways to inhibit biomaterial-related thrombosis.

### SUMMARY OF THE INVENTION

The present invention provides methods for targeting signal regulatory protein alpha (SIRP-α) to inhibit platelet binding and activation, with the overall goal of inhibiting platelet-mediated thrombotic events without compromising platelet function systemically.

An embodiment of the present invention provides a method for inhibiting a biomaterial-associated thrombotic event comprising reducing the number of platelets that bind to the biomaterial, and/or inhibiting platelet activation, by attaching CD47 or the Ig domain thereof to the surface of the biomaterial. For example, methods of the present invention inhibit thrombi formation on or near a biomaterial that is on the surface of an implant, medical device, tube, or therapeutic delivery vehicle.

Another embodiment of the present invention provides a biomaterial comprising CD47 or the Ig domain thereof attached to the surface of the biomaterial in an amount effective to reduce the number of platelets that bind to the biomaterial and to inhibit activation of the platelets. The invention also provides kits for inhibiting a biomaterial-associated thrombotic event comprising CD47 or the Ig domain thereof.

### BRIEF DESCRIPTION OF THE FIGURES

FIG. 1A: SIRP-α expression in human platelets as demonstrated by flow cytometry using anti-human SIRP-α antibody (SE7C2).

FIG. 1B: Representative immunostaining for human SIRP-α confirms SIRP-α expression in platelets.

FIG. 2A: Platelet attachment, assessed from whole blood sampled after a three hour-exposure to CD47-modified and control PVC tubing, via a Chandler Loop Apparatus, was significantly reduced as a result of CD47 exposure

FIG. 2B: Activation of non-attached platelets assessed from whole blood, sampled after a three hour-exposure to CD47-modified and control PVC tubing, using flow cytometry to detect platelet activation surface marker CD62P (* denotes P<0.01 compared to PVC).

### DETAILED DESCRIPTION OF THE INVENTION

Signal regulatory protein alpha (SIRP-α) is a transmembrane protein expressed in cells of myeloid origin, such as monocyte derived macrophages (MDMs). SIRP-α signaling is mediated by tyrosine inhibitory motifs (ITIMs) located in the cytoplasmic tail. The SIRP ITIMs activate Src homology domain 2-containing phosphatases-1 (SHP-1) and -2 (SHP-2). SIRP-α may also be identified as CD172A, SHPSl, P84, MYD-I, BIT, PTPNSl or SIRP-lα.

The applicants have surprisingly discovered that SIRP-α is expressed on the surface of platelets. SIRP-α has not previously been investigated in the context of platelets and the expression of SIRP-α in platelets has not been documented. Thus, targeting SIRP-α is a novel strategy to inhibit platelet binding and activation, with the overall goal of inhibiting platelet-mediated thrombotic events without compromising platelet function systemically. Embodiments of the present invention provide novel methods of inhibiting a biomaterial-associated platelet-mediated thrombotic event. In general, the methods comprise inhibiting platelet attachment and/or activation by attaching CD47, or the Ig domain thereof, to the surface of a biomaterial. CD47 immobilized surfaces have significant therapeutic potential in preventing platelet cell attachment and activation.

CD47, also known as integrin associated protein, is a ubiquitously expressed transmembrane protein. It is a member of the immunoglobulin (Ig) superfamily of membrane proteins with a single, variable Ig domain at its N terminus. The Ig domain of CD47 has been identified as a ligand of SIRP-α (Brown, E J et al. (2001) Trends Cell Biol 11: 130-5; Vernon-Wilson, E F et al. (2000) Eur. J. Immunol. 30:2130-7; Takizawa, H et al. (2007) Nat. Immunol. 8: 1287-9; and Subramanian, S et al. (2006) Blood 107:2548-56). As described in more detail below, the applicants have demonstrated that the interaction between SIRP-α and CD47 on the surface of biomaterials can confer thromboresistance to the biomaterials by reducing the number of platelets that adhere to the biomaterials, and by reducing platelet activation.

Platelets circulate in the blood and are involved in the formation of blood clots (also referred to as thrombi). Thrombosis is the formation of a blood clot inside a blood vessel, obstructing the flow of blood through the circulatory system. The formation of blood clots (thrombosis) may obstruct blood vessels and cause events such as stroke, myocardial infarction, pulmonary embolism, or the blockage of blood vessels to other parts of the body.

An embodiment of the present invention comprises a method for inhibiting a biomaterial-associated thrombotic event comprising reducing the number of platelets that bind to the biomaterial and/or inhibiting platelet activation (preferably both), by attaching CD47 or the Ig domain thereof to the surface of the biomaterial. A thrombotic event may comprise thrombosis or any other event in the body leading to, relating to, caused by, or characterized by thrombosis. Non-limiting examples of biomaterial-associated thrombotic events include aggregation of platelets, blood coagulation, thrombi formation (thrombosis) on or near the biomaterial, obstruction of blood flow through a blood vessel and/or medical device comprising the biomaterial (e.g., a blood conduit), and embolism.

Inhibiting a biomaterial-associated thrombotic event refers to entirely or substantially preventing a biomaterial-associated thrombotic event from occurring. Reducing the number of platelets that bind to a biomaterial comprising CD47 or the Ig domain thereof refers to reducing the number of platelets that bind to the biomaterial comprising CD47 or the Ig domain thereof compared to the number of platelets that bind to a control biomaterial (i.e., a biomaterial that does not comprise CD47 or the Ig domain thereof). For example, the number of platelets that bind to the biomaterial may be reduced by 100%, or by between about 10% to about 95%, between about 25% to about 90%, between about 50% to about 85%, or between about 70% to about 80% compared to a control biomaterial. The number of platelets that bind to a biomaterial comprising CD47 or the Ig domain thereof may be measured over the long-term or short-term (e.g., over the life of the biomaterial, immediately after a biomaterial is introduced to a subject's blood flow, or within hours or days after a biomaterial is introduced to a subject's blood flow, such as after about 1 hour, about 3 hours, about 12 hours, about 24 hours, or about 48 hours). Platelets that “bind” to a biomaterial include platelets that bind, attach, affix, or adhere to a biomaterial.

Similarly, inhibiting platelet activation by a biomaterial comprising CD47 or the Ig domain thereof refers to reducing the number of platelets that become activated compared to the number of platelets that become activated when a control biomaterial is used. This may be assessed, for example, by CD62P surface marker expression. CD62P, a platelet granule membrane protein, is expressed by the platelet surface upon platelet activation. For example, the activation of platelets by the biomaterial may be reduced by 100%, or by between about 10% to about 95%, between about 25% to about 75%, or between about 40% to about 60% compared to a control biomaterial. The number of platelets that become activated may be measured over the long-term or short-term, as described above.

Embodiments of the present invention provide biomaterials that are modified to reduce the number of platelets that bind to the biomaterial, and/or to inhibit activation of the platelets (preferably both). For example, a biomaterial of the present invention comprises CD47 or the Ig domain thereof attached to the surface of the biomaterial in an amount effective to reduce the number of platelets that bind to the biomaterial and/or to inhibit the activation of platelets. An “amount effective” includes any amount that reduces the number of platelets that bind to the biomaterial and/or that inhibit the activation of platelets, as compared to a control.

The methods described and exemplified herein are suitable for protecting any biomaterial. Biomaterials include any materials suitable for biological, biomedical, or medical applications. Non-limiting examples of biomaterials include fabrics, ceramics, polymers, thermoplastics such as polyaryletherketone and polyetherketoneketone, adhesives, bone cement, metals, and the like. Polymers are most preferred. Biomaterial polymers include, without limitation, polypropylene, polyethylene, polyester, polystyrene, polymethylmethacrylate, polyurethane, polyfluorotetraethylene, or polyvinyl (including polyvinyl chloride), polyethyleneimine, polyamide, polyacrylonitrile, polyacrylate, polymetacrylate, polyorthoester, polyether-ester, polylactone, polyalkylcyanoacrylate, polyethylenvinyl acetate, polyhydroxybutyrate, polytetrafluoroethylene, polyethylene terephthalate, polyoxyethylene, and the like, or mixtures thereof. Highly preferred polymers include polyurethane and polyvinyl chloride.

Biomaterials are used in various biological, biomedical, or medical applications. Such applications include compositions, products, and devices such as artificial joints, implants, stents, dental implants, bone cement, catheters, tubes, artificial tendons and ligaments, artificial skin, artificial heart valves, delivery vehicles for therapeutic agents, particles, and the like. In preferred aspects, the methods are applicable to protect the biomaterials used to fabricate these compositions, products, and devices and/or biomaterials coated onto the surface of these compositions, products, and devices. Thus, in preferred aspects, the methods are applicable to protect compositions, products, and devices comprising biomaterials. Preferably, the methods are used to protect implants, tubes, catheters, and therapeutic agent delivery vehicles comprising biomaterials. For example, the methods may be used to protect a device comprising the biomaterial selected from the group consisting of a stent (e.g., an intra-arterial stent), a catheter, a heart-lung bypass, a prosthetic heart valve, and a blood conduit. Compositions, products, and devices comprising biomaterials of the present invention may be for long-term use (such as a permanent implant), for short-term use (such as a temporary implant), or for temporary use, such as for during a procedure (e.g., angioplasty or cardiopulmonary bypass).

The described methods can utilize CD47, or any isoform thereof. The methods of the invention can also utilize the any suitable subdomain of CD47, including the extracellular Ig domain or subdomain thereof. Suitable subdomains of CD47 or its Ig domain preferably will be those that are capable of binding to or otherwise interacting with SIRP-α. The CD47, Ig domain, or other suitable subdomain thereof can be from any species, including mouse, rat, rabbit, horse, pig, sheep, cow, cat, dog, human and the like. Porcine, bovine and human CD47 are particularly preferred.

The methods described herein are suitable to reduce the number of platelets that bind to the biomaterial, and/or to inhibit platelet activation, in vitro, for example, biomaterials used in cell culture or in experiments generally. The methods are also suitable to reduce the number of platelets that bind to the biomaterial, and/or to inhibit platelet activation, in vivo, for example, biomaterials permanently or temporarily implanted, administered to, inserted, or otherwise inside of an animal.

The CD47, Ig domain, or suitable subdomain thereof can be attached to the biomaterial according to any means suitable in the art. For example, the CD47, Ig domain, or suitable subdomain thereof can be mixed with the biomaterial during manufacture of a composition, product, or device comprising the biomaterial such that the CD47, Ig domain, or suitable subdomain thereof is interspersed throughout the biomaterial, including on the surface of the particular composition, product, or device produced from the biomaterial. The CD47, Ig domain, or suitable subdomain thereof can alternatively be coated onto a portion of, or onto the entire, product or device. In some aspects, the CD47, Ig domain, or suitable subdomain thereof can be attached to the biomaterial, for example, by non-covalent intermolecular attractions, or by ionic or covalent bonds between the biomaterial and the CD47, Ig domain, or suitable subdomain thereof.

In some preferred aspects, the CD47, Ig domain, or suitable subdomain thereof can be attached to the biomaterial by way of linking molecules. The linking molecules can be complexed or conjugated to the biomaterial and/or the CD47, Ig domain, or suitable subdomain thereof. Linking molecules are any molecules capable of mediating or facilitating the attachment of the CD47, Ig domain, or suitable subdomain thereof to the biomaterial. Linking molecules can be any organic or inorganic chemical, proteins, polypeptides, polynucleotides, polysaccharides, lipids, thiols, and the like. Linking molecules are known in the art, and can be selected according to the needs of the practitioner. Some non-limiting examples of linking molecule pairs include avidin or streptavidin and biotin, thiol and Succinimidyl 3-(2-pyridyldithio)-propionate (SPDP) or Succinimidyl 4-[N-maleimidomethyl]cyclohexane-l-carboxylate (SMCC), or suitable variants or isoforms thereof, and folate and the folate receptor.

Embodiments of the invention include kits for protecting biomaterials utilizing the methods described and exemplified herein. In some aspects, the kits comprise CD47, the CD47 Ig domain, or subdomain thereof, and instructions for using the kit in a method for protecting a biomaterial. In some aspects, the kits further comprise one or more linking molecules capable of being attached to the surface of a biomaterial, and/or capable of being attached to the CD47, the CD47 Ig domain, or subdomain thereof, and may further comprise reagents suitable for attaching the linking molecule to the CD47, Ig domain, or subdomain thereof. In some aspects, the CD47, Ig domain, or subdomain thereof is complexed with a linking molecule.

The following examples are provided to describe embodiments of the invention in greater detail and are intended to illustrate, not limit, the invention.

### EXAMPLES

### Example 1

### SIRP-α is Expressed in Platelets

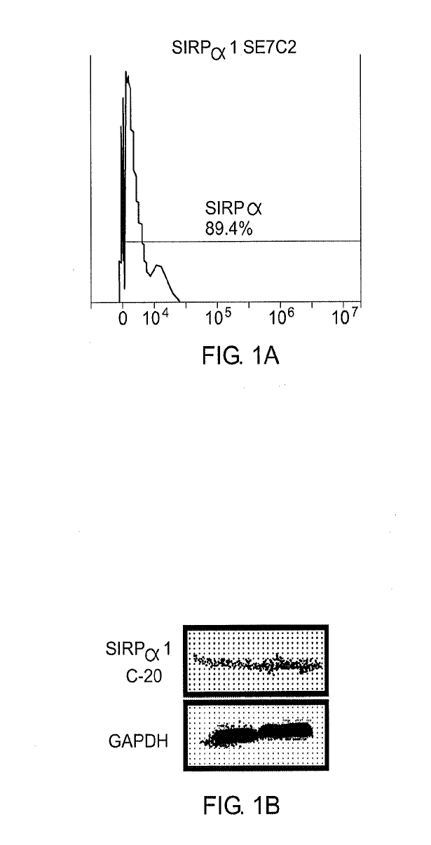

The following experiments examined SIRP-α expression in human platelets using flow cytometry and western blotting. Flow cytometry using magnetically purified platelets was completed using an antibody for S1RPa (SEC72) that binds to the extracellular domain of SIRP-α. As shown in FIG. 1A, nearly 90% of the purified platelets expressed SIRP-α on the surface.

Since membrane receptors can be shed into the blood serum and taken up by platelets, an antibody was tested against the intracellular SIRP-α domain. Purified platelets were lysed in a detergent-containing buffer and analyzed by western blotting. The SIRP-α (C-20) antibody exclusively measures intracellular SIRP-α and demonstrated a strong band at the correct molecular weight for SIRP-α (FIG. 1B). These findings suggest that a large percentage of platelets express SIRP-α.

### Example 2

### Platelet Activation and Attachment is Decreased When Whole Blood is Exposed to CD47-Modified Surfaces

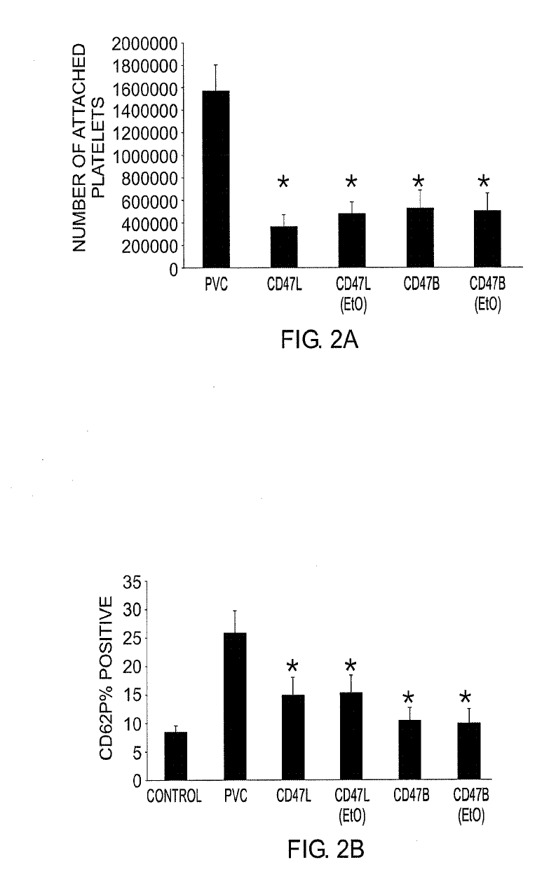

Human whole blood was exposed to polyvinyl chloride (PVC) modified with biotinylated CD47 (CD47B) or lysine tagged CD47 (CD47L). Following a three-hour exposure of human whole blood to the CD47-modified and control PVC tubing, attached blood cells were removed from the luminal surface using a EDTA-HEPES buffered solution (Tabuchi N, Shibamiya A, Koyama T, Fukuda T, Oeveren Wv W, Sunamori M. Activated leukocytes adsorbed on the surface of an extracorporeal circuit. Artif Organs. 2003;27(6):591-4), and assessed for the presence of attached and activated platelets. Where shown in FIGS. 2A and 2B, samples of tubing were sterilized by ethylene oxide (EtO). FIG. 2A shows that platelet attachment, from whole blood exposed to CD47-modified polymers via a Chandler Loop Apparatus, was significantly reduced as a result of CD47 exposure. Similarly, platelet activation (FIG. 2B) from unattached blood cells, as assessed by CD62P surface marker expression, was also significantly reduced. Similar results were noted for CD47 modified blood conduits that were sterilized via ethylene oxide exposure. These data demonstrate the hemocompatibility of CD47 modified surfaces, as well as their potential for clinical applications. Together, the findings above suggest that a large percentage of platelets express SIRP-α, and that the interaction between SIRP-α and CD47 on modified biomaterial surfaces may reduce the number of platelets adhered to the modified blood conduits.

Although the present invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the appended claims.

What is claimed is:

1. A method for inhibiting a biomaterial-associated thrombotic event comprising reducing the number of platelets that bind to the biomaterial by attaching CD47 or the Ig domain thereof to the surface of the biomaterial.

2. The method of claim 1, wherein the biomaterial-associated thrombotic event comprises thrombi formation on or near the biomaterial.

3. The method of claim 1, wherein the biomaterial comprises a polymer.

4. The method of claim 3, wherein the polymer is a polypropylene, polyethylene, polystyrene, polymethylmethacrylate, polyurethane, polyfluorotetraethylene, or polyvinyl, or mixtures thereof.

5. The method of claim 3, wherein the polymer is polyvinyl chloride.

6. The method of claim 3, wherein the polymer is polyurethane.

7. The method of claim 1, wherein the biomaterial is on the surface of an implant.

8. The method of claim 1, wherein the biomaterial is on the surface of a medical device.

9. The method of claim 1, wherein the biomaterial is on the surface of a tube.

10. The method of claim 1, wherein the biomaterial is on the surface of a therapeutic delivery vehicle.

11. The method of claim 7, wherein the implant comprises the biomaterial.

12. The method of claim 8, wherein the medical device comprises the biomaterial.

13. The method of claim 9, wherein the tube comprises the biomaterial.

14. The method of claim 10, wherein the therapeutic agent delivery vehicle comprises the biomaterial.

15. The method of claim 1, wherein a device comprising the biomaterial is selected from the group consisting of a stent, a catheter, a heart-lung bypass, a prosthetic heart valve, and a blood conduit.

16. The method of claim 1, wherein the surface of the biomaterial comprises at least one linking molecule.

17. The method of claim 16, wherein the CD47 or the Ig domain thereof is complexed with the linking molecule.

18. The method of claim 1, wherein the platelets express SIRP-α.

19. The method of claim 1, wherein the number of platelets that bind to the biomaterial is reduced by between about 10% to about 95% compared to a control biomaterial.

20. The method of claim 1, wherein the number of platelets that bind to the biomaterial is reduced by between about 50% to about 90% compared to a control biomaterial.

21. The method of claim 1, wherein the number of platelets that bind to the biomaterial is reduced by between about 70% to about 80% compared to a control biomaterial.

22. A method for inhibiting a biomaterial-associated thrombotic event comprising inhibiting platelet activation by attaching CD47or the Ig domain thereof to the surface of the biomaterial.

23. The method of claim 22, wherein the biomaterial-associated thrombotic event comprises thrombi formation on or near the biomaterial.

24. The method of claim 22, wherein the biomaterial comprises a polymer.

25. The method of claim 24, wherein the polymer is a polypropylene, polyethylene, polystyrene, polymethylmethacrylate, polyurethane, polyfluorotetraethylene, or polyvinyl, or mixtures thereof.

26. The method of claim 24, wherein the polymer is polyvinyl chloride.

27. The method of claim 24, wherein the polymer is polyurethane.

28. The method of claim 22, wherein the biomaterial is on the surface of an implant.

29. The method of claim 22, wherein the biomaterial is on the surface of a medical device.

30. The method of claim 22, wherein the biomaterial is on the surface of a tube.

31. The method of claim 22, wherein the biomaterial is on the surface of a therapeutic delivery vehicle.

32. The method of claim 28, wherein the implant comprises the biomaterial.

33. The method of claim 29, wherein the medical device comprises the biomaterial.

34. The method of claim 30, wherein the tube comprises the biomaterial.

35. The method of claim 31, wherein the therapeutic agent delivery vehicle comprises the biomaterial.

36. The method of claim 22, wherein a device comprising the biomaterial is selected from the group consisting of a stent, a catheter, a heart-lung bypass, a prosthetic heart valve, and a blood conduit.

37. The method of claim 22, wherein the surface of the biomaterial comprises at least one linking molecule.

38. The method of claim 37, wherein the CD47 or the Ig domain thereof is complexed with the linking molecule.

39. The method of claim 22, wherein the platelets express SIRP-α.

40. The method of claim 22, wherein the number of platelets activated by the biomaterial is reduced by between about 10% to about 95% compared to a control biomaterial.

41. The method of claim 22, wherein the number of platelets activated by the biomaterial is reduced by between about 25% to about 75% compared to a control biomaterial.

42. The method of claim 22, wherein the number of platelets activated by the biomaterial is reduced by between about 40% to about 60% compared to a control biomaterial.

43. A kit for inhibiting a biomaterial-associated thrombotic event, comprising CD47 or the Ig domain thereof and instructions for using the kit according to the method of claim 1.

44. The kit of claim 43, further comprising a linking molecule capable of being attached to the surface of the biomaterial.

45. A biomaterial comprising CD47 or the Ig domain thereof attached to the surface of the biomaterial in an amount effective to reduce the number of platelets that bind to the biomaterial and to inhibit activation of the platelets.

46. The biomaterial of claim 45, further comprising at least one linking molecule on the surface of the biomaterial.
